(19) United States
(12) Patent Application Publication
(10) Pub. No.: US 2007/0078505 A1
(43) Pub. Date: Apr. 5, 2007

(54) Two-balloon catheter device for coronary angioplasty and its use

(76) Inventor: Nicolay Grigorov Dimitrov, Sofia (BG)

Correspondence Address: KENYON & KENYON LLP, ONE BROADWAY, NEW YORK, NY, 10004, US

(21) Appl. No.: 11/399,054

(22) Filed: Apr. 5, 2006

(30) Foreign Application Priority Data
Apr. 5, 2005 (BG) 109111

(51) Int. Cl. A61F 2/06

(52) U.S. Cl. 623/1.11; 606/194; 604/101.05

(57) ABSTRACT

The invention relates to a two-balloon catheter device for coronary angioplasty and its use, which finds application in medicine for the treatment of stenosis (constriction or narrowing) of the coronary blood vessels. The two-balloon catheter device consists of a first balloon, monolithically fit into a first carrying catheter with a proximal and a distal end, a dilatation guide situated in the first balloon and in the first carrying catheter longitudinally and extending beyond the proximal and distal ends of the first catheter, a second balloon monolithically fit into a second carrying catheter with a proximal and a distal end. By means of a dilatation syringe with a nozzle, air is fed under pressure into the two catheters and the respective balloons, where the first carrying catheter is situated in the second balloon and in the second carrying catheter longitudinally and coaxially. Where the first carrying catheter is a dilatation guide of the second balloon and of the second carrying catheter. The two balloons are capable of two-way movement jointly or separately along the coronary blood vessel through the respective stenoses.

### FIELD OF THE INVENTION

The present invention relates to a two-balloon catheter device for coronary angioplasty and its use, which finds application in medicine for the treatment of stenosis (constriction or narrowing) of the coronary blood vessels.

### TECHNICAL BACKGROUND

From the technological background are known various kinds of balloon catheters used in coronary angioplasty.

Pat. EP 0820 784 B1 describes a conventional angioplasty balloon, which is a single balloon and can carry one stent, which stent is self-expanding.

U.S. Pat. No. 5,911,725 discloses a catheter used for retrieval of atherosclerotic plaques from the blood vessel, and not for dilatation of the latter. Therefore this catheter solves a substantially different problem from the objective of the present invention.

The task in U.S. Pat. No. 5,911,725 is to destroy the plaque in the blood vessel, whereas the aim of the two-balloon catheter device is to compress the plaque, i.e. to press it against the vessel wall.

The balloon known from US 2002/0049408 A1 is used for coronary dilatation, where additional metal fibers are preliminarily mounted on the balloon. There is a substantial difference between the construction of the known balloon and of the two-balloon catheter device.

The balloon catheters described in US 2002/0177889 A1 are designed for coronary dilatation, using additional energy such as vibrations for the breaking up of the atherosclerotic plaques.

In U.S. Pat. No. 4,924,863 the balloon is used to extract atherosclerotic plaques inside the coronary blood vessel, i.e. for aterectomy, whereas with the two-balloon catheter device the athero plaque is attacked in a different way in principle, namely by pressing it against the vessel walls, and not by scraping material off the plaque.

Pat. U.S. Pat. No. 4,808,164 discloses a balloon with chambers on its outer surface and offers an additional source of action on the plaques.

Pat. U.S. Pat. No. 6,039,721 describes a balloon, which is used as drug carrier, where the purpose of the drugs is to dissolve or soften the plaque.

Pat. U.S. Pat. No. 6,485 500 B1 discloses an embolization protection system, comprising one or more blocking balloons. Unlike the two-balloon catheter device for coronary angioplasty, the balloons are fixed to their carrier, i.e. they are not movable with respect to each other and move together in the blood vessel lumen. This presupposes that they cannot cover two coronary plaques, which are at a distance different from the preset distance of the fixed balloons. Whereas in the two-balloon catheter device the distance between the balloons is not fixed and it can be adjusted depending on the anatomy of the different patients.

U.S. Pat. No. 6,156,005 discloses a balloon catheter for stent implantation, consisting of one occlusive balloon fixed to a catheter by means of a horizontal tube-shaped part. A dilatation balloon is fixed on the catheter, which is essentially a therapeutic balloon. The described balloon catheters are used for implanting a stent, which may be non-self-expanding or self-expanding. The balloon catheter comprises one or more dilatation balloons, which do not move freely and two-way with respect to each other.

### SUMMARY OF THE INVENTION

An aim of the present invention is to create a two-balloon catheter device for coronary angioplasty, which would ensure dilatation of two high degree stenoses of the coronary blood vessel of more than 75% of the vessel diameter, which are located consecutively, within the same operative time. An aim of the invention is by the use of a two-balloon catheter device for coronary angioplasty to ensure dilatation of two high degree stenoses of the coronary blood vessel of more than 75% of the vessel diameter, which are located consecutively, within the same operative time. An aim of the invention is also to reduce the risk of rupture of the blood vessel by means of a single introduction of a two-balloon catheter device into the vessel, which leads to higher rate of success in restoring the patency and blood flow of the affected blood vessel, as well as to reduction of the exposure time for operator and patient.

An advantage of the two-balloon catheter device compared to the hitherto used balloon catheters is that it reduces the risk of rupture of the coronary blood vessel in the dilatation of stenoses, reduces the risk of acute thrombosis of the coronary blood vessel or obstruction of the same, while at the same time the rate of success in restoring the patency and blood flow of the coronary blood vessel is increased. Another advantage of the invention is that dilatation is done in twice as little operative time, since two stenoses are dilated by a single introduction of the two-balloon catheter device into the blood vessel.

An advantage of the invention is also that by reducing the operative time the exposure of operator and patient is also decreased.

### DESCRIPTION OF THE PRESENT INVENTION

The two-balloon catheter device for coronary angioplasty consists of a first balloon, first carrying catheter, dilatation guide, a second balloon, a second carrying catheter, and one or two dilatation syringes with a nozzle. The first balloon is monolithically fit into the first carrying catheter, having a proximal end and a distal end. The dilatation guide represents a thin metal wire and is situated longitudinally in the first balloon and in the first carrying catheter. The dilatation guide extends out of the proximal end and the distal end of the first carrying catheter. The second balloon is monolithically fit into the second carrying catheter, having a proximal end and a distal end. The first carrying catheter is situated in the second balloon and in the second carrying catheter longitudinally and coaxially at its proximal end. In this way, the first carrying catheter essentially functions as dilatation guide of the second balloon and of the second carrying catheter. The first carrying catheter and the first balloon and the second carrying catheter and the second balloon are capable of two-way movement jointly or separately along the coronary blood vessel, reaching and passing through the respective stenoses. By means of one or two dilatation syringes with nozzles, air is fed under pressure into the first carrying catheter and the first balloon and respectively into the second carrying catheter and the second balloon. In order to ensure the free movement of the balloons with respect to each other together with the respective catheters, the second carrying catheter is of larger diameter than the first carrying catheter, respectively the second balloon has a larger diameter than the first balloon. Stents can be preliminarily mounted on the first and second balloons. A stent may be mounted only on the first balloon or only on the second balloon.

The use of the two-balloon catheter device for coronary angioplasty described above provides consecutive dilatation of two high degree stenoses of a coronary blood vessel within the same operative time by a single introduction of the device into the vessel for dilatation of stenoses and implantation of one or two stents. The first balloon is used for dilatation of the coronary blood vessel and/or as support for the second balloon, and the second balloon is used as support for the first balloon and/or for dilatation of the coronary blood vessel, where a stent is preliminarily mounted only on one of the balloons or on both balloons. The two-balloon catheter device for coronary angioplasty is used for dilatation of high degree stenoses of the coronary blood vessel of more than 75% of the vessel diameter.

### BRIEF DESCRIPTION OF THE DRAWINGS

FIG. 1 shows the proposed two-balloon catheter device for coronary angioplasty using one dilatation syringe.

FIG. 2 shows the proposed two-balloon catheter device with stents mounted on the first and second balloon, using one dilatation syringe.

FIG. 3 shows the proposed two-balloon catheter device with a stent mounted only on the first balloon, using one dilatation syringe.

FIG. 4 shows the proposed two-balloon catheter device with a stent mounted only on the second balloon, using one dilatation syringe.

FIG. 5 shows the proposed two-balloon catheter device for coronary angioplasty using two dilatation syringes.

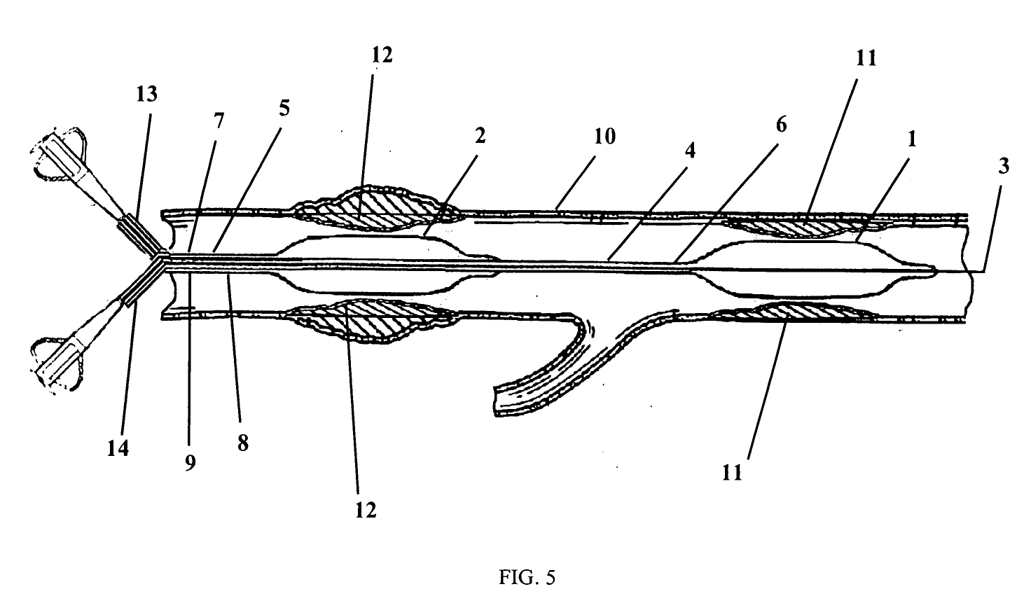

FIG. 6 shows the proposed two-balloon catheter device with stents mounted on the first and second balloon, using two dilatation syringes.

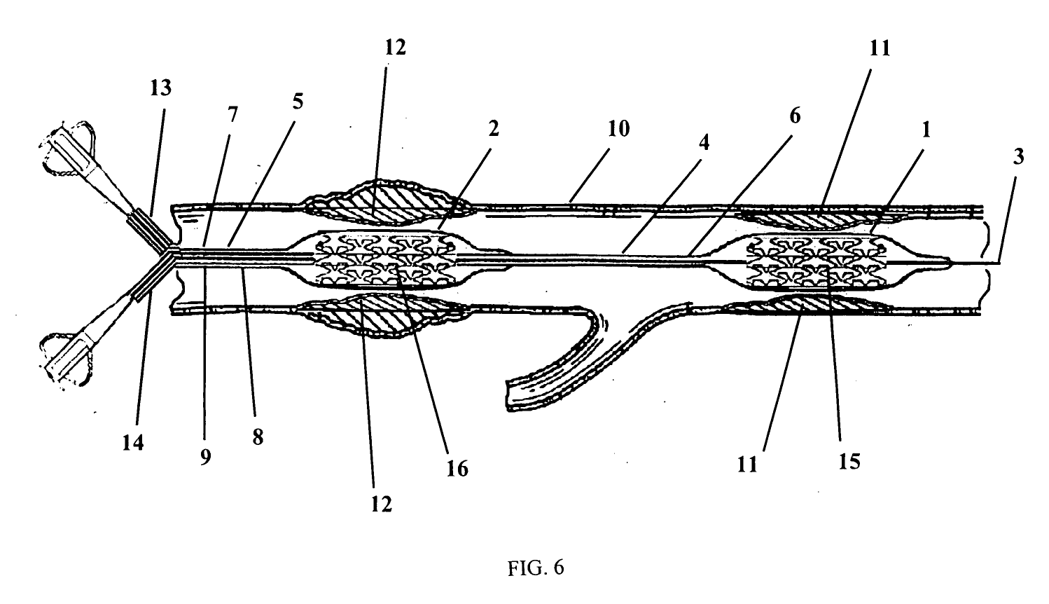

FIG. 7 shows the proposed two-balloon catheter device with a stent mounted only on the first balloon, using two dilatation syringes.

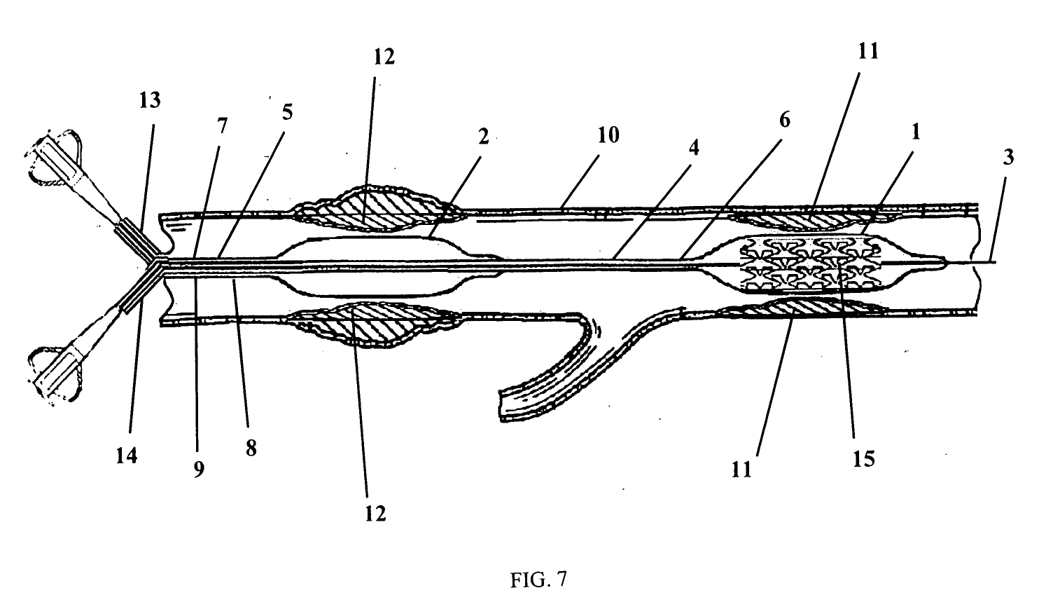

FIG. 8 shows the proposed two-balloon catheter device with a stent mounted only on the second balloon, using two dilatation syringes.

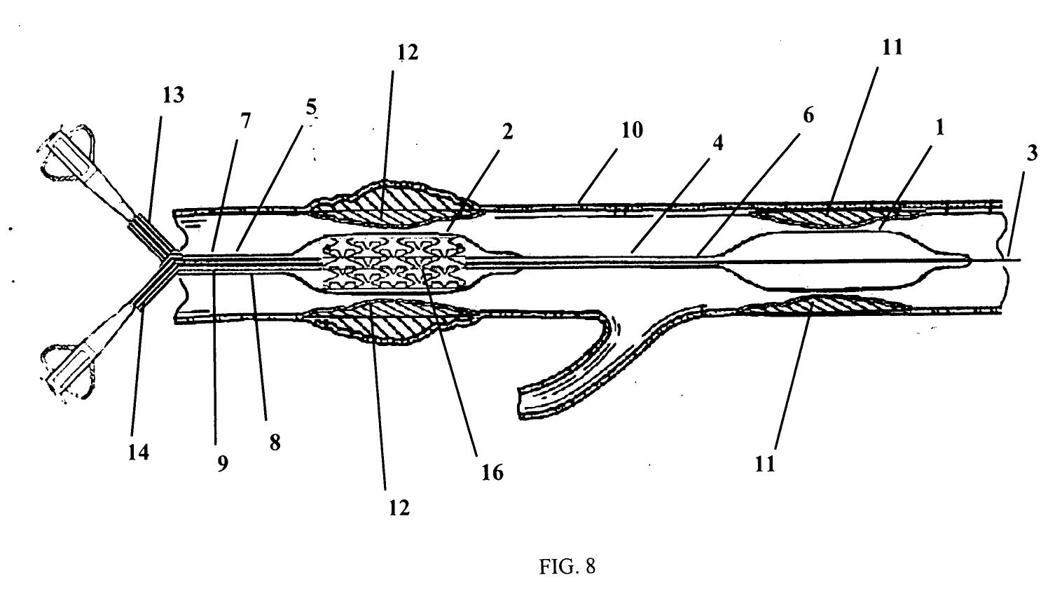

### EXAMPLES OF EMBODIMENT OF THE INVENTION

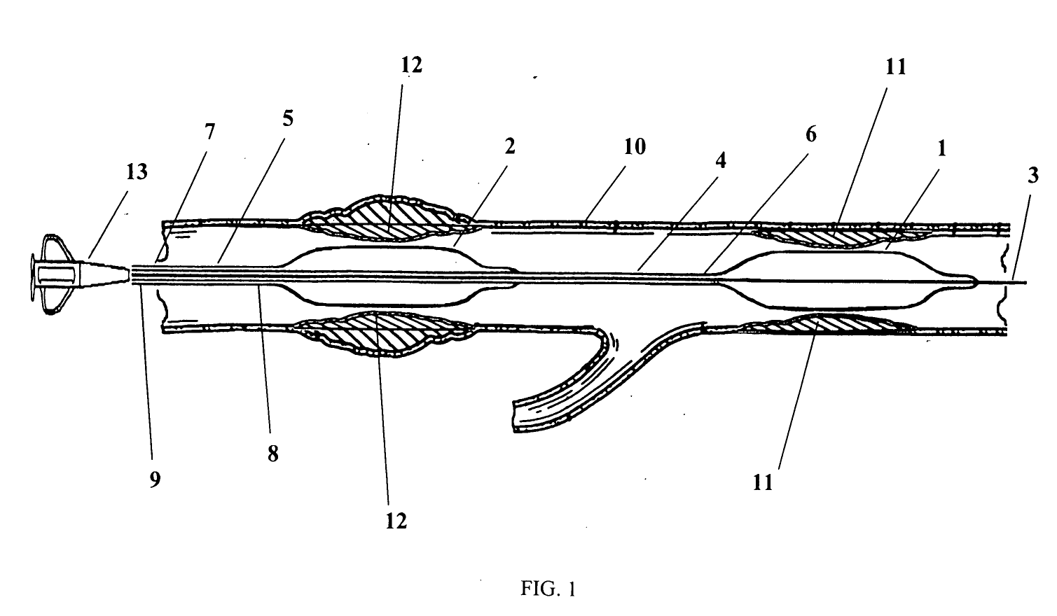

According to the present invention, the two-balloon catheter device for coronary angioplasty shown in FIG. 1 consists of a first balloon 1 and a second balloon 2, which are fit monolithically in a first carrying catheter 4 and a second carrying catheter 5, respectively. Along the entire length of the first carrying catheter 4 and through the first balloon 1 passes a dilatation guide 3, which extends out of the proximal and the distal ends of the first carrying catheter 4. The dilatation guide 3 is a thin metal wire. The first carrying catheter 4 is situated longitudinally and coaxially in the second balloon 2 and in the second carrying catheter 5 at the proximal end of the second catheter. The two catheters are capable of free two-way movement of each catheter or both catheters simultaneously. The first carrying catheter 4 is essentially a dilatation guide of the second balloon 2 and the second carrying catheter 5. The dilatation guide 3 moves freely two-way in the first carrying catheter 4 and in the first balloon 1. The dilatation guide 3 extending beyond the proximal part of balloon 1 facilitates the passing of balloon 1 through stenosis 11 of the blood vessel 10. Balloon 1 is positioned with respect to stenosis 11 so as to cover the whole length of the stenosis. Balloon 2 is positioned with respect to a second stenosis 12 of the blood vessel 10, which is adjacent to stenosis 11. After the positioning of the two balloons air under pressure is fed into them simultaneously by means of a dilatation syringe mounted by a nozzle to the distal ends of each of the two carrying catheters 4 and 5 of the device, which are situated outside the blood vessel. The two-balloon catheter device provides an opportunity to restore the patency of the coronary blood vessel 10 and the blood flow through two consecutive stenoses 11, 12 in twice as short operative time, where the risk of rupture of the coronary blood vessel is reduced multiply due to the single introduction of the device into the blood vessel 10. After the stenoses are dilated, by means of a control-recording device the restored patency of the blood vessel 10 is established and verified, and the air in the two balloons is exhausted by means of a dilatation syringe 13. Then a new recording of the restored blood vessel patency is made.

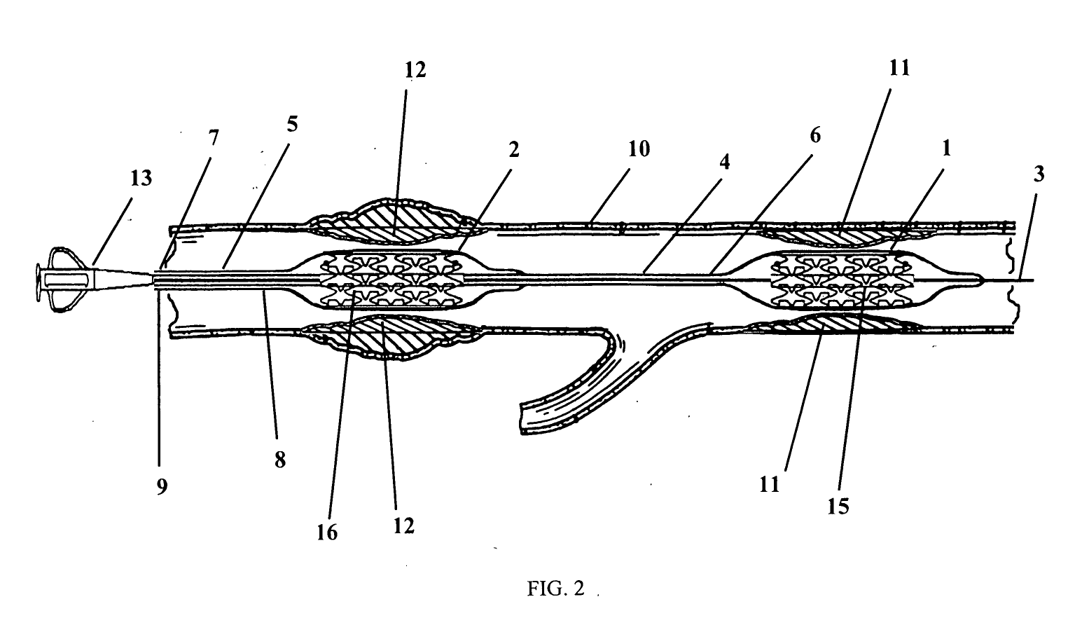

In one embodiment of the invention, shown in FIG. 2, on the first balloon 1 and the second balloon 2 of the two-balloon catheter device stents 15 and 16 are factory mounted. After the air exhaustion, the stents remain implanted in the blood vessel 10 at the location of the stenoses 11, 12.

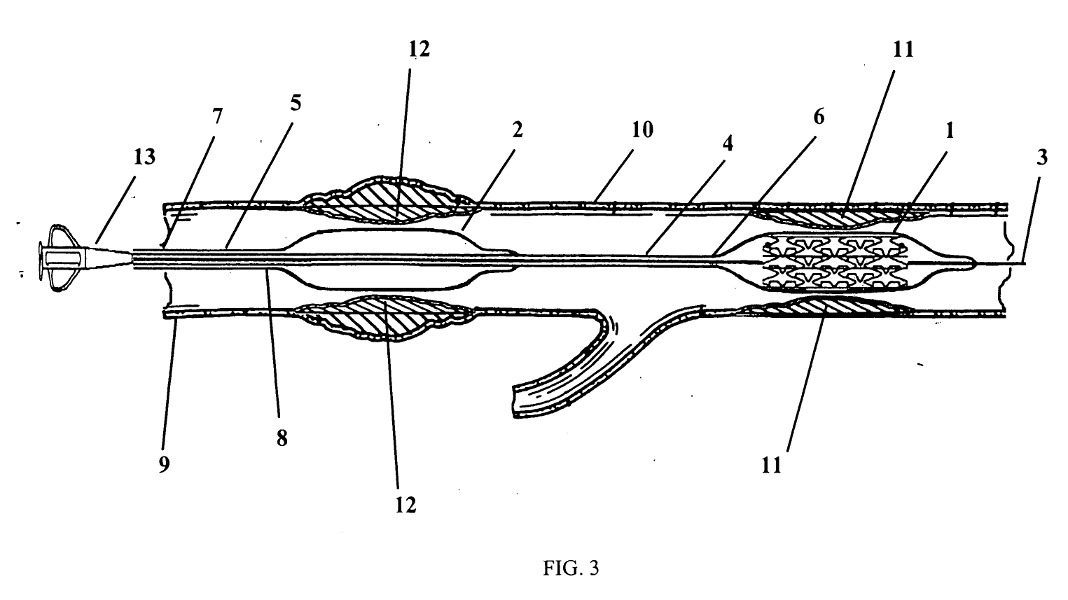

In another embodiment of the invention, shown in FIG. 3, only on the first balloon 1 of the two-balloon catheter device a stent 15 is factory mounted. After the air exhaustion, the stent 15 remains implanted in the blood vessel 10 at the location of the stenosis 11.

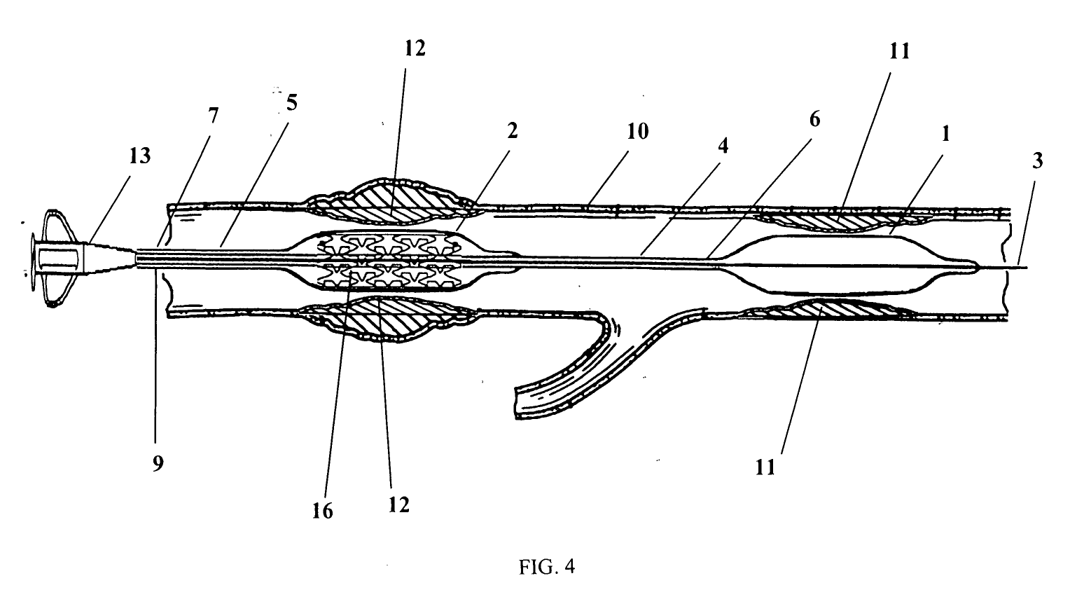

In a further embodiment of the invention, shown in FIG. 4, only on the second balloon 2 of the two-balloon catheter device a stent 16 is factory mounted. After the air exhaustion, the stent 16 remains implanted in the blood vessel 10 at the location of the stenosis 12.

The use of the two-balloon catheter device described in Example 1 is made by dilating two consecutively located high degree stenoses 11 and 12 in the same coronary blood vessel 10 within the same operative time by a single introduction of the device into the vessel 10, where the first balloon 1 is used for dilatation of stenosis 11 in the coronary blood vessel 10 and/or as support for the second balloon 2, and the second balloon 2 is used as support for the first balloon 1 and/or for dilatation of stenosis 12 in the coronary blood vessel 10, where a stent 11, 12 is preliminarily mounted only on one of the balloons 1, 2 or on both balloons.

1-9. (canceled)

10. A device comprising:
(a) a first balloon catheter;
(b) a dilatation guide disposed within the first balloon catheter and extending past a distal end of the first balloon catheter;
(c) a second balloon catheter disposed about the first balloon catheter, supporting the first balloon catheter, and moveable relative to the first balloon catheter; and
(d) at least one syringe adapted to inflate and deflate at least one of a first balloon on the first balloon catheter and a second balloon on the second balloon catheter,
wherein the first balloon catheter supports the second balloon catheter and the first balloon and the second balloon are capable of two-way movement one of jointly and separately.

11. The device according to claim 10, wherein the at least one syringe comprises a single syringe adapted to simultaneously inflate the first balloon and the second balloon and to simultaneously deflate the first balloon and the second balloon.

12. The device according to claim 10, wherein the at least one syringe comprises a first syringe adapted to inflate and deflate the first balloon and a second syringe adapted to inflate and deflate the second balloon.

13. The device according to claim 10, wherein at least one of (i) the first balloon and the first balloon catheter are monolithic and (ii) the second balloon and the second balloon catheter are monolithic.

14. The device according to claim 10, wherein the at least one syringe includes a nozzle.

15. The device according to claim 10, wherein the first balloon catheter and second balloon catheter are coaxial.

16. The device according to claim 10, further comprising a nozzle connected to the at least one syringe.

17. The device according to claim 10, wherein the at least one syringe is adapted to feed air under pressure into the first balloon and the second balloon.

18. The device according to claim 10, wherein the dilatation guide is a metal wire.

19. The device according to claim 10, further comprising at least one stent mounted on at least one of the first balloon and the second balloon.

20. The device according to claim 10, wherein the first balloon and second balloon are dilatation balloons adapted to dilate coronary blood vessel stenoses.

21. The device according to claim 20, wherein the stenoses occupy more than 75% of a diameter of the blood vessel.

22. A method for using a two-balloon catheter device, comprising:
(a) inserting the two-balloon catheter device into a patient such that a first balloon on the two-balloon catheter device lies within a first stenosis in a blood vessel of the patient; and
(b) one of simultaneously and consecutively inflating the first balloon and a second balloon on the two-balloon catheter device within the same operative time so as to one of simultaneously and consecutively dilate the first stenosis and a second stenosis in the blood vessel within the same operative time by a single introduction of the two-balloon catheter device into the blood vessel.

23. The method according to claim 22, prior to step (b) moving the second balloon relative to the first balloon such that the second balloon on the two-balloon catheter device lies within the second stenosis in the blood vessel of the patient.

24. The method according to claim 22, wherein a stent is mounted on at least one of the first balloon and the second balloon and further comprising the steps of deflating the first balloon and the second balloon.

25. The method according to claim 23, wherein the first stenosis and the second stenosis each occupy more than 75% of a diameter of the blood vessel.
